(19) United States
(12) Patent Application Publication
(10) Pub. No.: US 2021/0369352 A1
(43) Pub. Date: Dec. 2, 2021

(54) ULTRASONIC WAVE IMAGING APPARATUS, THERAPY SUPPORT SYSTEM, AND IMAGE DISPLAY METHOD

(71) Applicant: HITACHI, LTD., Tokyo (JP)

(72) Inventors: Hirozumi TAKESHIMA, Tokyo (JP); Tomohiko TANAKA, Tokyo (JP); Ryo IMAI, Tokyo (JP)

(21) Appl. No.: 17/233,904

(22) Filed: Apr. 19, 2021

(30) Foreign Application Priority Data
May 29, 2020 (JP) 2020-094987

(51) Int. Cl. A61B 34/20; A61B 8/08; A61B 8/12; A61B 8/00; G16H 30/40; G16H 20/40

(52) U.S. Cl. CPC A61B 34/20; A61B 8/0841; A61B 8/12; A61B 8/4218; A61B 8/4488; A61B 2090/3788; G16H 30/40; G16H 20/40; A61B 2034/2063; A61B 2034/2065; A61B 8/466

(57) ABSTRACT

An ultrasonic wave imaging apparatus is disclosed, including: an ultrasonic probe for irradiating a subject with an ultrasonic wave and receive a reflected wave of the ultrasonic wave and receiving an ultrasonic wave from a beacon inserted into the subject; a probe position-acquiring unit for acquiring a 3D position and an orientation of the ultrasonic probe; a beacon location-acquiring unit for determining a 3D location of the beacon from relative location and speed of the beacon relative to the ultrasonic probe as calculated from an ultrasonic wave image received at the ultrasonic probe and the 3D position and the orientation of the ultrasonic probe as acquired by the probe position-acquiring unit; and a display image formation section for using the ultrasonic wave image of the ultrasonic waves from the ultrasonic probe to form a display image. A corresponding method is also disclosed.

### TECHNICAL FIELD

The present invention relates to an ultrasonic wave imaging apparatus for capturing an ultrasonic wave image by inserting, in biomedical tissue, a guidewire equipped with, for instance, a photoacoustic ultrasonic wave generator; a therapy support system; and an image display method.

### BACKGROUND ART

Catheterization has been widely and primarily used for treatment such as stenosis because a patient's burden in this operation is less than in surgery such as thoracotomy. It is critical to grasp the relationship between a treatment target area and a catheter during catheterization, so that X-ray fluoroscopy has been used as an imaging support method. In addition, JP 2019-213680A discloses that an ultrasound image was attempted to be used as a support image instead of X-ray fluoroscopic image.

Specifically, JP 2019-213680A discloses a technology in which regarding an ultrasonic wave generated by an ultrasonic wave generator installed on a guidewire, an arrival time difference occurring when the ultrasonic wave (the ultrasonic wave from the ultrasonic wave generator) arrives at an element array included in an ultrasonic probe or an ultrasonic wave generator image depending on the distance in an imaging area is used to estimate the tip position of the guidewire; and the estimation results are then used to grasp the relative positional relationship between the imaging output and the guidewire tip.

### SUMMARY OF INVENTION

### Technical Problem

The above previous technology makes it possible to estimate the location of the tip position of an insert (guidewire) in the imaging area. Unfortunately, to grasp the 3D positional relationship between a living body imaging target such as a blood vessel and the insert tip, a 2D-array probe is required in which element arrays constituting an ultrasonic probe are arranged like a matrix.

In addition, although the relative positional relationship of, for instance, a catheter relative to an imaging area can be grasped, the absolute position cannot be detected. Consequently, the positioning on in vivo information acquired by, for instance, another imaging technique is impossible.

The purpose of the present invention is to provide an ultrasonic wave imaging apparatus, a therapy support system, and an image display method such that a linear array probe can used to grasp the 3D positional relationship between a guidewire and an imaging target such as a blood vessel.

### Solution to Problem

An aspect of the present invention provides an ultrasonic wave imaging apparatus comprising:

an ultrasonic probe configured to irradiate a subject with an ultrasonic wave and receive a reflected wave of the ultrasonic wave and receive an ultrasonic wave from a beacon inserted into the subject;

a probe position-acquiring unit configured to acquire a 3D position and an orientation of the ultrasonic probe;

a beacon location-acquiring unit configured to determine a 3D location of the beacon from a relative location and a relative speed of the beacon relative to the ultrasonic probe as calculated from an ultrasonic wave image of the ultrasonic waves received at the ultrasonic probe and the 3D position and the orientation of the ultrasonic probe as acquired by the probe position-acquiring unit; and

a display image formation section configured to use the ultrasonic wave image of the ultrasonic waves received at the ultrasonic probe to form an image displayed on a display unit.

### Advantageous Effects of Invention

According to the invention, a linear array probe can be used to teach a surgeon about the 3D positional relationship between a guidewire and an imaging target such as a blood vessel.

### BRIEF DESCRIPTION OF DRAWINGS

FIG. 1 is a diagram illustrating the overall configuration of a medical support system in an embodiment.

FIG. 2 is a diagram illustrating how the tip of a guidewire looks like.

FIG. 3 is a block diagram for an ultrasonic wave imaging apparatus.

FIG. 4 is a flowchart of processing in the ultrasonic wave imaging apparatus.

FIG. 5 is a diagram illustrating a coordinate system for an ultrasonic probe.

FIG. 6 is a diagram illustrating how to detect the absolute position of a PA signal generator from changes in position of an ultrasonic probe and a PA signal generator.

FIG. 7 is a diagram illustrating how to detect the absolute position of a PA signal generator from a change in rotational position of an imaging plane of an ultrasonic probe.

FIG. 8 is a flowchart of processing for detecting the absolute position of a PA signal generator by using the speed of an ultrasonic probe.

FIG. 9 is a block diagram for an ultrasonic wave imaging apparatus with another configuration.

FIG. 10 is a flowchart of another processing in the ultrasonic wave imaging apparatus.

FIG. 11 is a diagram showing a example of display on a display unit.

FIG. 12 is a diagram showing another example of display on the display unit.

FIG. 13A is a diagram showing an example of how to plan movement of a robot arm by an operation planning section.

FIG. 13B is a diagram showing an example of ultrasonic wave image from an ultrasonic probe.

FIG. 14A is a diagram showing another example of how to plan movement of the robot arm by the operation planning section.

FIG. 14B is a diagram showing another example of ultrasonic wave image from the ultrasonic probe.

### DESCRIPTION OF EMBODIMENTS

Hereinafter, embodiments of the invention will be described in detail with reference to the Drawings.

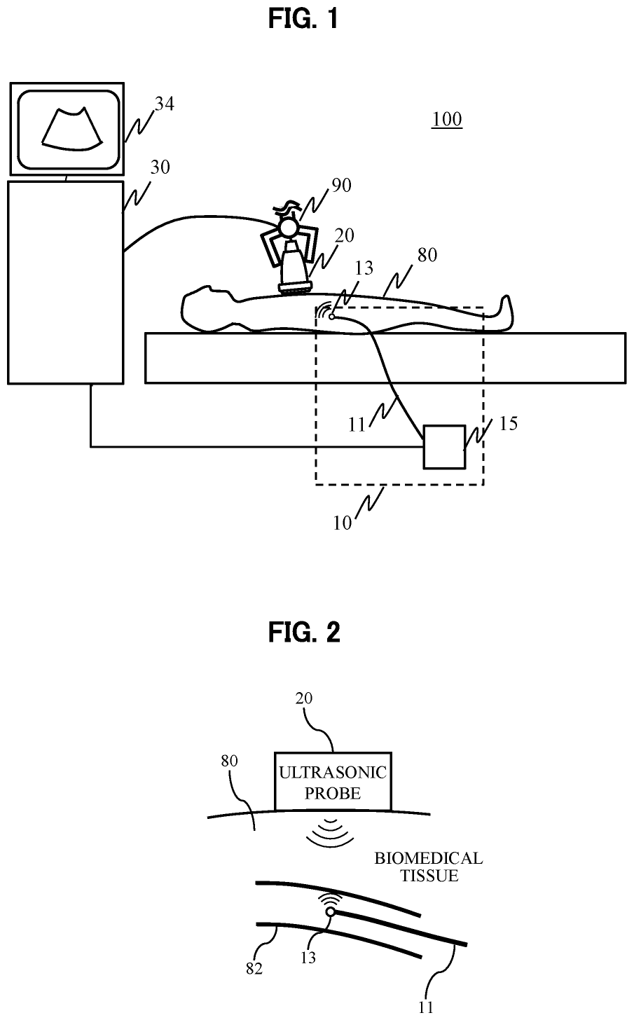

FIG. 1 is a diagram illustrating an ultrasonic wave imaging apparatus according to an embodiment of the invention and the overall configuration of a catheterization support system (hereinafter, sometimes referred to as a medical support system) using the apparatus.

Here, FIG. 2 is a diagram illustrating how the tip of a guidewire looks like.

As shown in FIG. 1, the medical support system 100 includes: a body insertion instrument (guidewire) 11 equipped with an ultrasonic wave-generating device 10 including an ultrasonic wave generator (beacon) 13 and a photogeneration module 15; an ultrasonic probe (probe) 20; a robot arm 90; and an ultrasonic imaging module 30 for acquiring an ultrasonic wave image of a subject 80 into which the body insertion instrument 11 has been inserted, and a display unit 34 thereof.

Examples of the body insertion instrument 11 include long and thin tubular medical devices such as balloon catheters, microcatheters, nutritional catheters, and other therapeutic devices as well as guidewires for delivering each therapeutic device to a target site. A case where the body insertion instrument is a guidewire is described below.

The following describes an ultrasonic wave-generating device 10 provided with a PA signal generator 13 that uses an photoacoustic (PA) effect to generate an ultrasonic wave signal. However, the ultrasonic wave may be generated by a piezoelectric element.

As shown in FIGS. 1 and 2, the ultrasonic wave-generating device 10 includes: an optical fiber 12 (not shown) positioned inside a hollow portion of a flexible, hollow guidewire 11; the photoacoustic ultrasonic wave generator 13 fixed to an insertion-side end face of the optical fiber 12; and a photogeneration module 15 that is connected to the other end (an end opposite to the end fixed to the photoacoustic ultrasonic wave generator 13) of the optical fiber 12 and generates a laser beam. The optical fiber 12 functions as a beam-guiding member for guiding a laser beam generated by the photogeneration module 15 to the photoacoustic ultrasonic wave generator 13. These ultrasonic wave-generating device 10 and hollow guidewire 11 are sometimes together called a photoacoustic source-equipped wire.

FIG. 2 shows that the ultrasonic probe 20 is used to detect an ultrasonic wave generated by the photoacoustic ultrasonic wave generator 13 (hereinafter, referred to as a PA signal generator 13) at the tip of the guidewire 11 inserted into a blood vessel 82. Then, the ultrasonic imaging module 30 superimposes an image of the detected PA signal generator 13 on a cross sectional image of the subject 80 or the blood vessel 82 as created on the basis of an ultrasonic wave emitted from the ultrasonic probe 20.

This makes it possible to grasp the location of the guidewire 11 in the subject 80 or the blood vessel 82.

The PA signal generator 13 is made of material that can be subject to adiabatic expansion upon reception of a laser beam to generate an ultrasonic wave such as a PA signal. Examples of the material include a known pigment (photosensitizer), metal nanoparticles, or a carbon-based compound. The tip of the optical fiber 12 including the PA signal generator 13 is covered with a resin sealing member. Note that in FIG. 2, the PA signal generator 13 is positioned at the tip of the guidewire 11. However, the position is not limited to the wire tip.

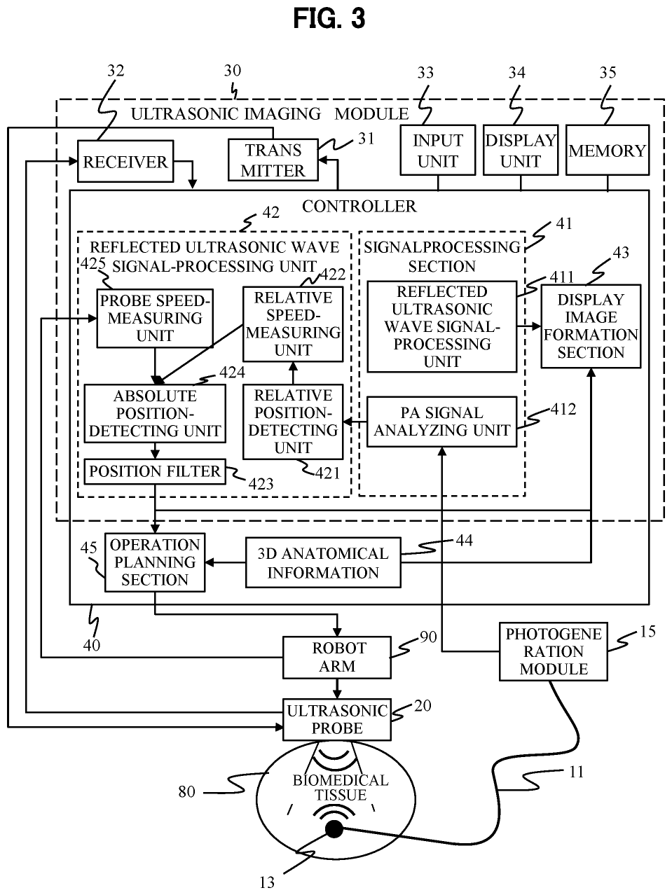

Next, the ultrasonic imaging module 30 as a component of the ultrasonic wave imaging apparatus of this embodiment will be described in detail. FIG. 3 is a block diagram for the ultrasonic imaging module 30 included in the ultrasonic wave imaging apparatus.

The ultrasonic imaging module 30 includes: a controller 40 detailed later; a transmitter 31 for transmitting an ultrasonic wave signal to the ultrasonic probe 20; a receiver 32 for receiving a reflected wave (RF signal) detected at the ultrasonic probe 20 to perform, for instance, phasing and/or addition processing; an input unit 33 for inputting an instruction and/or conditions required for imaging by a user; a display unit 34 for displaying, for instance, an ultrasonic wave image acquired by the ultrasonic imaging module 30 and/or graphic user interface (GUI); and a memory 35 for storing, for instance, a processing output and/or a display image formed, based on the processing output, by a display image formation section 43.

The controller 40 includes: a signal-processing section 41 configured to process an ultrasonic wave image (including a reflected ultrasonic wave signal and a PA signal) received at the ultrasonic probe 20; a PA source location detection section 42 configured to use a signal processed by the signal-processing section 41 to detect the source location of the PA signal; a display image formation section 43 configured to form an image displayed on the display unit 34 while using the PA source location detected by the PA source location detection section 42 and pre-acquired 3D anatomical information 44 about a subject as obtained beforehand; and an operation planning section 45 configured to use the source location detected by the PA source location detection section 42 and the pre-acquired 3D anatomical information 44 about the subject as obtained beforehand to determine an operation position of the ultrasonic probe 20.

The pre-acquired 3D anatomical information 44 used may be a 3D volume image obtained by CT (Computer Tomography) and/or MRI (Magnetic Resonance Imaging) or a 3D volume image captured by sweeping with the ultrasonic wave imaging apparatus. The signal-processing section 41 includes: a reflected ultrasonic wave signal-processing unit 411 configured to use the RF signal, which is a reflected wave received by the receiver 32, to create an ultrasonic wave image such as a B-mode image; and an ultrasonic signal analyzing unit (PA signal analyzing unit) 412 configured to detect and process, based on the beam-emitting timing of the laser beam from the photogeneration module 15, a PA signal that is generated from the PA signal generator 13 and is then detected at each transducer element of the ultrasonic probe 20.

Note that the controller 40 is configured like a common ultrasonic wave imaging apparatus except for addition of the PA signal analyzing unit 412 where the PA signal is received and the PA source location detection section 42 configured to detect the location where the PA signal is generated.

The PA source location detection section 42 includes: a relative position detecting unit 421 configured to estimate, from the PA signal analyzed by the PA signal analyzing unit 412, the location of the PA signal generator 13 in an imaging area; a relative speed-measuring unit 422 configured to derive the speed by differentiating the location of the PA signal generator 13 as detected by the relative speed-measuring unit 421; a probe speed-measuring unit 425 configured to measure, based on operation position information about the robot arm 90, the speed of the ultrasonic probe 20; an absolute position-detecting unit 424 configured to detect the absolute location of the PA signal generator 13 on the basis of the speed measured by the relative speed-measuring unit 422 and the speed of the ultrasonic probe 20 as measured by the probe speed-measuring unit 425; and a position filter 423 used to reduce an error by filtering the absolute position detected by the absolute position-detecting unit 424.

In the position filter 423, any filtering may be used to reduce an error included in the detection results of the absolute position-detecting unit 424. For instance, in the case where the PA signal generator 13 moves at a low speed, an error during the position detection may be reduced by smoothening with a filter such as a movement averaging filter or low pass filter (LPF).

In addition, the position filter 423 may be a filter in view of an error model configured to model a detection error that can occur, in principle, in a position detection technique of the PA source location detection section 42. Specifically, the location and speed of the PA signal generator 13 as detected in the PA source location detection section 42, the position, speed, and attitude of the ultrasonic probe 20, and an error model for the PA source location detection section 42 may be integrally considered to apply a Kalman filter and/or a particle filter that can infer the statistically most likely state.

The details of the other configurations in the PA source location detection section 42 will be described later.

Part or all of the functions of the controller 40 may be implemented by executing software with a program(s) for the functions in a computer provided with a CPU(s) or GPU(s) and a memory. In addition, part or all of the functions of each unit may be implemented using hardware such as an electronic circuit, ASIC, or FPGA. Note that the controller 40 may be installed at a single computer or the functions may be separately installed at a plurality of computers.

The ultrasonic probe 20 may be a 1D-array probe (linear array probe) having one array sequence of multiple transducer elements aligned in a 1D direction. In addition, various kinds of the ultrasonic probe 20 may be used, including a 3D-array probe having 2 or 3 array sequences in directions perpendicular to an array sequence direction of the 1D array probe; or a 2D-array probe having multiple array sequences in 2D directions. The signal-processing section 41 employs an analysis technique depending on the type of the ultrasonic probe 20 used.

Next, how the medical support system 100 in this embodiment works will be described.

Here, the ultrasonic wave imaging apparatus is configured such that while the ultrasonic probe 20, which is a 1D-array probe, is used to capture an ultrasonic wave from the subject 80, the guidewire 11 (e.g., a catheter) that is guided by a surgeon and has, at the tip, the PA signal generator 13 is inserted into the body of a subject; and the ultrasonic wave imaging apparatus then monitors the tip location of the guidewire 11 by using the PA signal. The following describes a case of operating the robot arm 90 such that the ultrasonic probe 20 tracks the tip location of the guidewire.

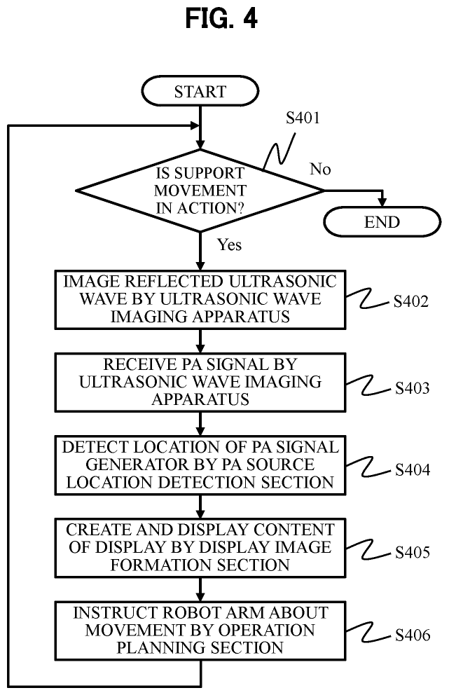

FIG. 4 is a flowchart of processing in the ultrasonic wave imaging apparatus.

At step S401, the ultrasonic wave imaging apparatus determines whether or not a support operation for tracking the ultrasonic probe 20 is in action. If not in action (S401: No), the processing is ended. If in action (S401: Yes), the processing goes to step S402.

At step S402, the ultrasonic wave imaging apparatus uses the ultrasonic probe 20 to capture a reflected ultrasonic wave (hereinafter, referred to as an imaging mode). In this imaging mode, an ultrasonic wave is captured like in conventional ultrasonic wave imaging apparatuses.

Specifically, the transmitter 31 transmits an ultrasonic wave through the ultrasonic probe 20 and the ultrasonic probe 20 receives a reflected wave after the transmitted ultrasonic wave is reflected from a tissue inside a subject. The receiver 32 performs phasing/addition processing of the reception signal that is received, as each frame, from the ultrasonic probe 20, and send the results to the signal-processing section 41. The reflected ultrasonic wave signal-processing unit 411 uses the frame signal from the receiver 32 to create an ultrasonic wave image such as a B-mode image, and transfer the image to the display image formation section 43 configured to form an image displayed on the display unit 34.

In this imaging mode, if the ultrasonic probe 20 is a 1D-array probe, it is possible to obtain information about the intensity of the reflected wave in the array probe direction and the depth direction. The information about the intensity of the reflected wave can be used to acquire 2D information about the intensity of the reflected wave.

Meanwhile, in the case of using a 2D-array probe as the ultrasonic probe 20, the imaging mode may be performed just once to obtain 3D information corresponding to the intensity of the reflected wave together in the probe plane and depth directions.

At step S403, the ultrasonic wave imaging apparatus uses the ultrasonic probe 20 to receive a PA signal (hereinafter, referred to as a PA reception mode). This PA reception mode can be used to monitor a PA signal from the PA signal generator 13 when a catheter is being inserted into the body (e.g., a blood vessel) of the subject.

Specifically, during the PA reception mode, the operation of the transmitter 31 is temporarily stopped, and the photogeneration module 15 is actuated to emit a pulsed laser beam from the photogeneration module 15. The PA signal generator 13 is irradiated, through the optical fiber 12 of the guidewire 11 inserted into the body, with the beam emitted by the photogeneration module 15. This irradiation beam causes a PA signal (ultrasonic wave) to occur from the photoacoustic material of the PA signal generator 13. Then, the PA signal is detected by elements of the ultrasonic probe 20.

The PA signal analyzing unit 412 use the PA signal received at the ultrasonic probe 20 to prepare signal data synchronized with the beam emitted from the photogeneration module 15. Then, the data is transferred to the relative position detecting unit 421 in the PA source location detection section 42. To synchronize the received PA signal, each timing may be obtained from a trigger signal output to the PA signal analyzing unit 412 upon emission of the beam from the photogeneration module 15. Alternatively, each beam emission timing may be inferred from the PA signal received by the elements of the ultrasonic probe 20.

In addition, in the case of using the ultrasonic wave-generating device 10 configured to generate an ultrasonic wave by using a piezoelectric element, the transmitter 31 may be used to transmit the ultrasonic wave, so that the signal generating timing can be inferred. Also, an external signal source and the trigger signal may be used for the synchronization like in the case of using the PA signal generator.

At step S404, the PA source location detection section 42 detects the location of the PA signal generator 13 on the base of information about the PA signal transferred from the PA signal analyzing unit 412 and the absolute (3D) position and the attitude (orientation) of the ultrasonic probe 20 as sent from the robot arm 90.

Specifically, in the PA source location detection section 42, first, the relative position detecting unit 421 detects the relative location of the PA signal generator 13 relative to the ultrasonic probe 20 on the basis of the PA signal transferred from the PA signal analyzing unit 412.

Next, the relative speed-measuring unit 422 detects the relative speed from a temporal change in the relative position detected. Then, the probe speed-measuring unit 425 detects the speed and angular velocity of the ultrasonic probe 20 from a temporal change in information about the absolute position and attitude (orientation) of the ultrasonic probe 20 as sent from the robot arm 90.

In addition, the relative speed-measuring unit 422 may measure the relative speed by using a Doppler effect occurring in the received PA signal.

After that, the absolute position-detecting unit 424 uses these values to detect the absolute location of the PA signal generator 13.

The detected absolute location of the PA signal generator 13 is filtered with the position filter 423 to reduce a detection error.

At step S405, the display image formation section 43 forms a display content to be displayed on the display unit 34.

Specifically, the display image formation section 43 uses a reflected ultrasonic wave image formed by the reflected ultrasonic wave signal-processing unit 411, the location of the PA signal generator 13 as detected by the PA source location detection section 42, and a 3D volume image regarding the anatomical structure of the subject 80 as obtained beforehand and recorded on the pre-acquired 3D anatomical information 44 to form a display image to be displayed on the display unit 34 in such a manner as to enable a surgeon to understand the 3D positional relationship of the PA signal generator 13.

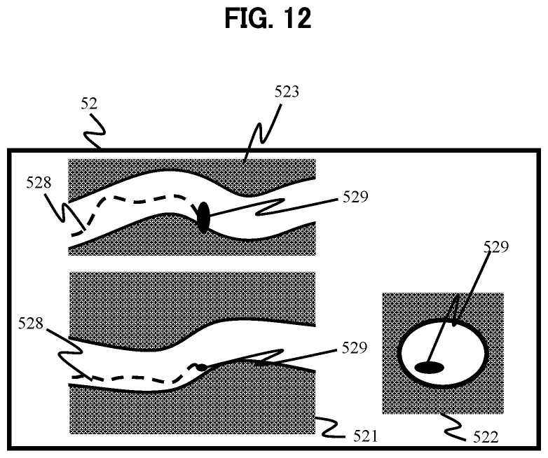

FIGS. 11 and 12 later describe specific examples displayed on the display unit 34.

At step S406, the operation planning section 45 uses the pre-acquired 3D anatomical information 44 about the subject 80, the location and coordinates of the probe as obtained from the robot arm 90, and the reflected ultrasonic wave image and the PA source location acquired by the ultrasonic wave imaging apparatus to plan an operation of the robot arm 90 and then instruct the robot arm 90 about the operation position.

For instance, the operation planning section 45 permits the robot arm 90 to move such that the PA signal generator 13 is positioned at a given position on the reflected ultrasonic wave image at step S402 so as to track the PA signal generator 13. This makes it possible for the ultrasonic probe 20 to continue receiving the PA signal (ultrasonic wave) generated from the PA signal generator 13.

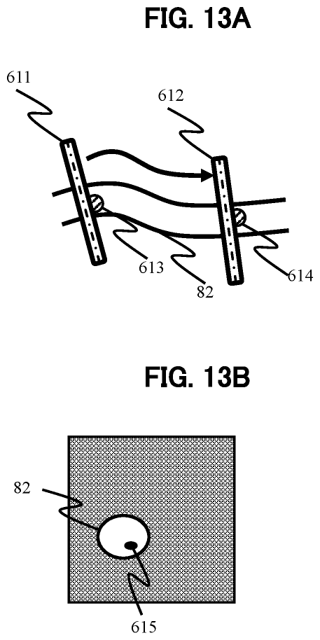

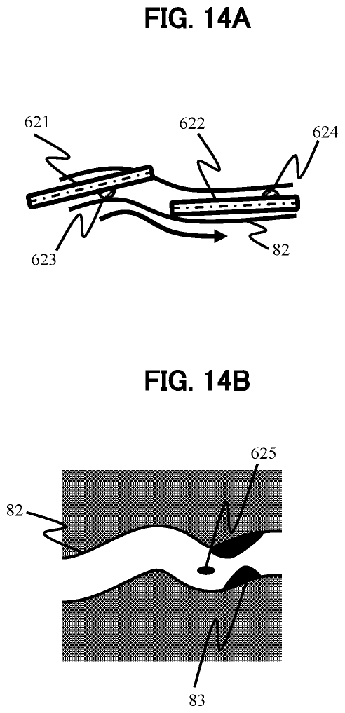

FIGS. 13A and 14A later describe more specific examples of tracking by the ultrasonic probe 20.

Subsequently, the processing returns to step S401, and steps S402 to 406 are then repeated.

Note that the imaging mode (step S402) and the PA reception mode (step S403) are not limited to those in the flowchart of FIG. 4. For instance, a cycle of operation, in which the imaging mode is executed four times and the PA reception mode is executed once, may be repeated; or a cycle of operation, in which the imaging mode and the PA reception mode are each executed once, may be repeated.

Hereinafter, processing of the absolute position-detecting unit 424 at step S404 in FIG. 4 will be described in detail.

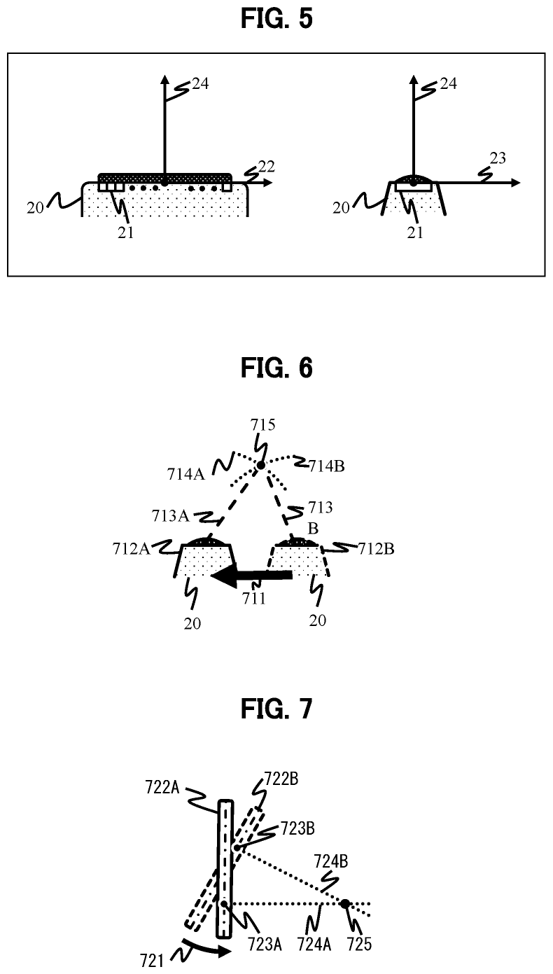

FIG. 5 is a diagram illustrating, for the description below, a coordinate system for the ultrasonic probe 20. In the coordinate system, the major axis 22 is set to the array alignment direction of the photoacoustic element array 21 in the ultrasonic probe 20, which is a 1D-array probe; the minor axis 23 is set to an axis parallel to the array reception plane and perpendicular to the major axis 22; the depth axis 24 is set to an axis normal to the array reception plane; and the origin of the coordinate system is set to the point of intersection between the major axis and the minor axis on the surface plane of the photoacoustic element array 21.

If the ultrasonic probe 20 is a 2D-array probe with multiple array sequences in the 2D direction, the orientation of the major axis 22 or the minor axis 23 may be determined arbitrarily.

First, FIG. 6 illustrates how to determine the relationship between the position and the attitude when the ultrasonic probe 20 is translated in the direction of minor axis 23, that is, how to detect the absolute location of the PA signal generator 13 from changes (during translation) in position of the PA signal generator 13 and the ultrasonic probe 20.

As shown in FIG. 6, if the ultrasonic probe 20 is a 1D-array probe and the ultrasonic probe 20 is translated (711) from a position 712B to a position 712A in the direction of minor axis 23, the absolute position of the ultrasonic probe 20 at each absolute position (712A or 712B) can be acquired from the robot arm 90. In addition, since the time from the beam emission at the photogeneration module 15 to the arrival of the PA signal at the ultrasonic probe 20 can be estimated by the PA signal analyzing unit 412, the distance 713A or 713B from the position 712A or 712B to the PA signal generator 13 can be determined, respectively.

Thus, since the position 715 of the PA signal generator 13 is a point of intersection between the arc 714A of the distance 713A and the arc 714B of the distance 713B, the absolute location can be calculated from the absolute position (712A or 712B) of the ultrasonic probe 20 and the distance (713A or 713B) to the PA signal generator 13, respectively.

Specifically, the distance 713A (lₐ) or 713B (l_{b}) to the PA signal generator 13 can be determined using formula (1) where tₐ or t_{b} is set to the time of arrival of the PA signal before or after the movement.

[Formula 1]

l_{b}=c·t_{b}, lₐ=c·tₐ (1)

where c is the sound speed.

Then, if the ultrasonic probe 20 is translated from the position 712B to 712A at a speed v in a small time period Δt, the position 715 (y_{PA} or z_{PA}) of the PA signal generator 13 can be determined using formula (2).

$$\begin{matrix}[ Formula 2 ] & \\ {y}_{P A} = \frac{{l}_{b} - {t}_{a}}{v \cdot Δ t} {l}_{a} , {z}_{P A} = \sqrt{{l}_{a}^{2} - {y}_{P A}^{2}} & ( 2 )\end{matrix}$$

Provided that y represents a relative value in the direction of minor axis 23; z represents a relative value in the direction of depth axis 24; and approximation lₐ≈l_{b} is made because the small time period Δt is sufficiently short.

In the above case, the PA signal generator 13 rests. However, it may be moved. This causes an error in the absolute location of the PA signal generator 13 as calculated from the movement of the PA signal generator 13. Here, the position filter 423 may be used to reduce the error.

Next, FIG. 7 illustrates how to determine the relationship between the position and the attitude when the ultrasonic probe 20 rotates about the depth axis 24, that is, how to detect the absolute location of the PA signal generator 13 from a change in rotational position of the imaging plane of the ultrasonic probe 20.

FIG. 7 shows the case where the ultrasonic probe 20, which is a 1D-array probe, rotates about the depth axis 24 from the position 722B to 722A in the direction of rotation 721. In this case, the PA signal analyzing unit 412 can acquire the position 723B or 723A of the PA signal generator 13 in the direction of major axis; and the absolute position at the position 723B or 723A of the PA signal generator 13 in the direction of major axis, respectively, can then be acquired from the robot arm 90.

Since the location 725 of the PA signal generator 13 is a point of intersection between the straight line 724A and the straight line 724B, the absolute positions 723A and 723B can be used to calculate the absolute position at the location 725 of the PA signal generator 13.

Specifically, when the change x_{PA} in position of the PA signal generator 13 in the direction of major axis 22 between before and after the movement, the angular velocity ω of the rotation 721 about the depth axis 24, the arrival time t of the PA signal, and the sound speed c are set, the location 725 (y_{PA} or z_{PA}) of the PA signal generator 13 can be determined using formula (3).

$$\begin{matrix}[ Formula 3 ] & \\ {y}_{P A} = \frac{d {x}_{P A}}{d t} / ω , {z}_{P A} = \sqrt{{( t \cdot c )}^{2} + {y}_{P A}^{2}} & ( 3 )\end{matrix}$$

In the method for detecting the absolute location of the PA signal generator 13 from the changes in position of the above ultrasonic probe 20 and the PA signal generator 13 or the change in rotational position of the imaging plane of the ultrasonic probe 20, the positions before and after the movement are used to detect the absolute location of the PA signal generator 13. However, if the time difference between before and after the movement is small, the speed of the ultrasonic probe 20 may be used to detect the absolute location of the PA signal generator 13.

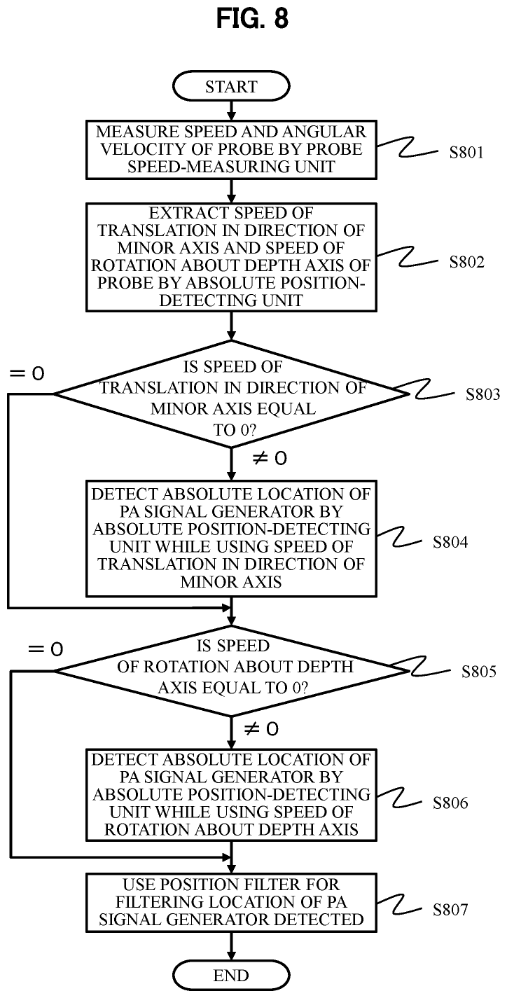

FIG. 8 is a flowchart illustrating processing for detecting, in the PA source location detection section 42, the absolute location of the PA signal generator 13 by using the speed of the ultrasonic probe 20.

In this flowchart, the translation and/or the rotation are extracted from the movement of the ultrasonic probe 20 to detect the absolute location of the PA signal generator 13.

At step S801, the probe speed-measuring unit 425 measures the movement speed and angular velocity of the ultrasonic probe 20. Here, the movement speed and angular velocity of the ultrasonic probe 20 may be acquired from the robot arm 90.

At step S802, the absolute position-detecting unit 424 uses the movement speed and angular velocity of the ultrasonic probe 20 to extract the translation speed of the ultrasonic probe 20 in the direction of minor axis 23 and the speed component of the rotation of the ultrasonic probe 20 about the depth axis 24.

At step S803, the absolute position-detecting unit 424 determines whether or not the extracted translation speed in the direction of minor axis 23 is equal to 0. If equal to 0 (S803: “=0”), the processing goes to step S805. If unequal to 0 (S803: “not 0”), the processing goes to step S804.

At step S804, like the method described in FIG. 6, the absolute position-detecting unit 424 uses each translation speed of the PA signal generator 13 or the ultrasonic probe 20 in the direction of minor axis 23 to detect the absolute location of the PA signal generator 13. Specifically, the absolute location of the PA signal generator 13 is detected such that the PA signal generator 13 is located at a point with an equal distance from the two points with a distance obtained at the translation speed for the speed measurement period. Then, the processing goes to step S805.

At step S805, the absolute position-detecting unit 424 determines whether or not the extracted speed of rotation of the ultrasonic probe 20 about the depth axis 24 is equal to 0. If equal to 0 (S805: “=0”), the processing goes to step S807. If unequal to 0 (S805: “not 0”), the processing goes to step S806.

At step S806, like the method described in FIG. 7, the absolute position-detecting unit 424 uses the speed of rotation of the ultrasonic probe 20 about the depth axis 24 to detect the absolute location of the PA signal generator 13. Specifically, the absolute location of the PA signal generator 13 is detected such that the PA signal generator 13 is located at a point of intersection between the normal lines at the two points with a distance obtained at the rotation speed for the measurement period. Then, the processing goes to step S807.

At step S807, the position filter 423 is used to filter at least one of the absolute locations of the PA signal generator 13 as calculated at step S804 and step S806. This can refine the position detection.

Hereinabove, the case of using a 1D-array probe as the ultrasonic probe 20 has been described. Here, the case of using a 2D-array probe as the ultrasonic probe 20 will be described.

In this case, the 3D location of PA signal generator 13 relative to the ultrasonic probe 20 can be obtained without acquiring the 3D location of the PA signal generator 13 from information about the translation and/or rotation of the ultrasonic probe 20. Here, the acquired 3D relative location may be added to the 3D absolute position and attitude of the ultrasonic probe 20 as notified from the robot arm 90 to detect the 3D absolute location of the PA signal generator 13.

The above ultrasonic wave imaging apparatus and therapy support system have been used to describe that the robot arm 90 operates the ultrasonic probe 20 and the 3D absolute location of the PA signal generator 13 can be detected based on the absolute position of the ultrasonic probe 20 detected by the robot arm 90. It may be configured to provide a probe position/attitude sensor 91 for detecting the absolute position of the ultrasonic probe 20. In addition, the robot arm 90 may be used to operate the ultrasonic probe 20, and the probe position/attitude sensor 91 may be used to detect the absolute position of the ultrasonic probe 20.

The probe position/attitude sensor 91 may be built in the ultrasonic probe 20 or may be configured as a separate body.

The probe position/attitude sensor 91 may be configured by combining, for instance, a positioning sensor (e.g., a geomagnetic sensor), an accelerometer, and/or a gyrometer, or may use an external camera. This sensor is not limited if the position and attitude of a probe can be measured.

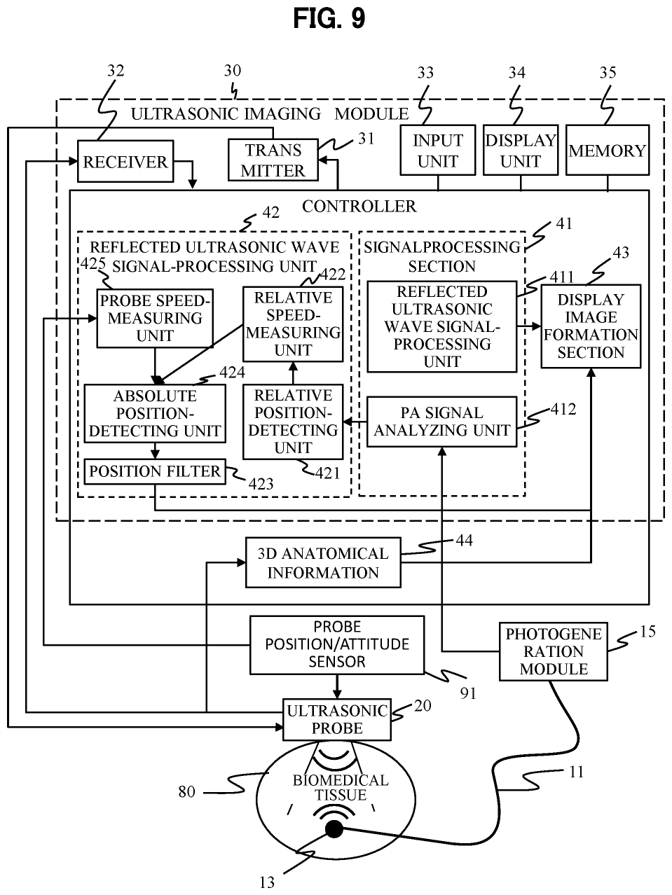

FIG. 9 is a block diagram illustrating the structure of an ultrasonic wave imaging apparatus and a therapy support system configured to detect the absolute position of the ultrasonic probe 20 by using the probe position/attitude sensor 91.

The ultrasonic wave imaging apparatus in FIG. 9 has the same configuration as of the ultrasonic wave imaging apparatus described in FIG. 3 except that the operation planning section 45 for the robot arm 90 is excluded and the probe position/attitude sensor 91 is included.

The probe position/attitude sensor 91 is configured to periodically detect the absolute position and attitude of the ultrasonic probe 20 and notify the controller 40 about them.

Then, the probe speed-measuring unit 425 detects the speed and angular velocity of the ultrasonic probe 20 from a temporal change in information about the absolute position and attitude (orientation) of the ultrasonic probe 20 as sent from the probe position/attitude sensor 91 instead of the robot arm 90.

The display image formation section 43 uses the absolute position of the ultrasonic probe 20 as detected by the probe position/attitude sensor 91 to perform substantially the same processing as in the case of detecting the 3D absolute location of the PA signal generator 13.

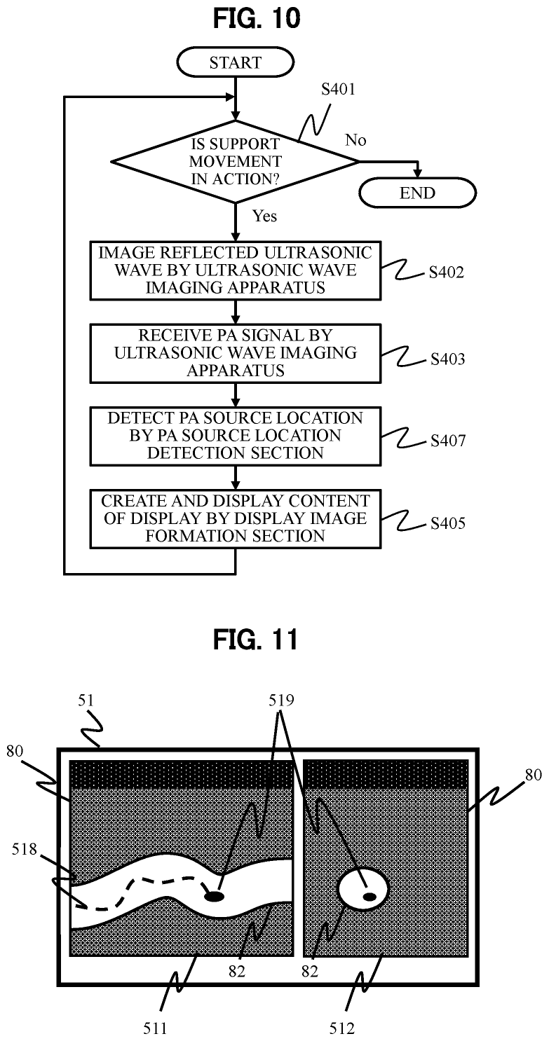

FIG. 10 is a flowchart of processing in the ultrasonic wave imaging apparatus.

The differences from the flowchart of processing in the ultrasonic wave imaging apparatus described in FIG. 4 involve points where the processing for instructing the robot arm about its operation by the operation planning section at step S406 is excluded and step S404 is replaced by step S407.

At step S407, the PA source location detection section 42 detects the location of the PA signal generator 13 on the basis of information about the PA signal transferred from the PA signal analyzing unit 412 and the (3D) absolute position and attitude (orientation) of the ultrasonic probe 20 as sent from the probe position/attitude sensor 91.

This enables the 3D absolute location of the PA signal generator 13 to be detected even in the case of manually operating the ultrasonic probe 20.

Hereinabove, the embodiments for detecting the location of the PA signal generator 13 have been described. However, the respective embodiments may be used in combination, if appropriate, as long as they are technically consistent, and such a combination should be included in the invention.

Here, described are display images displayed on the display unit 34 at step S405 in FIG. 4 or 10.

FIG. 11 is a diagram showing an example of display image 51 displayed on the display unit 34.

As shown in FIG. 11, the display image 51 displayed on the display unit 34 is used to show the location of the PA signal generator 13 in the blood vessel 82 of the subject 80, and includes an image 511, in which a major axis cross-section of the blood vessel 82 is displayed, and an image 512, in which a transverse section of the blood vessel 82 is displayed. Then, the body and the blood vessel 82 of the subject 80 and the location mark 519 and the tracking path 518 of the PA signal generator 13 are displayed on the image 511 or 512. All or part of these display elements may be displayed.

In addition, the image 511 and the image 512 may be displayed using each display image obtained by imaging by the ultrasonic imaging module 30 and then formed by the display image formation section 43. Also, the images may be formed by computer graphics (CG) using information about the PA signal generator 13 detected by the PA source location detection section 42 and the pre-acquired 3D anatomical information 44 about the subject 80 as obtained beforehand. In this case, the display image formation section 43 may display information about the anatomical structure outside the imaging area of the ultrasonic imaging module 30.

The details of the image displayed on the display unit 34 may include any processed image to support surgery and may include a display, in which a notable point such as a blood vessel or a lesion present in the above ultrasonic wave image or the CG is emphasized, and/or a display, in which a site of lesion outside the screen is indicated.

During the image formation by means of the CG, the pre-acquired 3D anatomical information 44 about the subject 80 as obtained beforehand may be used to detect where is the position on the absolute coordinates as detected by the robot arm 90 for positioning. For this purpose, features in the ultrasonic wave image formed by the display image formation section 43 and features in the pre-acquired 3D anatomical information 44 are compared to detect any agreement point. Examples of the features that can be used include blood vessel branches and/or bones as well as other features.

In addition, during the image formation by means of the CG, the pre-acquired 3D anatomical information 44 may be used directly to form the display image. Alternatively, the display image may be formed by using information about the pre-acquired 3D anatomical information 44 as obtained beforehand and then modified such that deformation of the anatomical structure during surgery from the pre-acquired anatomical structure is detected and the pre-acquired 3D anatomical information 44 as obtained beforehand is fit better to the structure during surgery. For instance, it is possible to consider modification processing in which a blood vessel wall that has been formed during surgery and is in a reflected ultrasonic wave image is recognized; and a blood vessel wall included in the pre-acquired 3D anatomical information 44 as obtained beforehand is fit likewise to have a shape of the blood vessel wall in the reflected ultrasonic wave image.

The content of the image displayed on the display unit 34 is not limited to a screen image displayed in two directions as exemplified in FIG. 11. An image in one direction may be displayed or an image may be displayed by a third angle projection method if a 3D positional relationship between a lesion and the PA signal generator 13 can be presented. In addition, four or more image screens from any eye points may be displayed. Here, the display method and the eye points for the displayed image are not limited.

FIG. 12 is a diagram showing an example of display image 52 displayed by a third angle projection method.

The display image 52 includes a blood vessel lateral view 521, a blood vessel transverse view 522, and a blood vessel top view 523, which are images in three directions.

Reference sign 528 in FIG. 12 denotes a tracking path of the PA signal generator 13. Reference sign 529 denotes the location of the PA signal generator 13.

The following details how to instruct movement of the robot arm, which is planed by the operation planning section 45 at step S406 in FIG. 4.

FIGS. 13A and 14A each illustrate an example of a plan for movement of the robot arm 90 by the operation planning section 45.

In FIG. 13A, the robot arm 90 displaces the ultrasonic probe 20 such that when the PA signal generator 13 is moved from the pre-movement location 613 to the post-movement location 614, the ultrasonic probe 20 is moved from the pre-movement position 611 to the post-movement position 612 while the imaging area of the ultrasonic probe 20 is kept to give a transverse view of the blood vessel 82, so that the PA signal generator 13 is tracked.

FIG. 13B is an ultrasonic wave image when the robot arm 90 is used to move the ultrasonic probe 20 like in FIG. 13A.

In the ultrasonic wave image, the blood vessel 82 of the subject 80 and the PA signal generator 13 (location 615) are depicted. This makes it easier to grasp the location 615 of the PA signal generator 13 in the blood vessel 82 of the subject 80.

In FIG. 14A, the robot arm 90 displaces the ultrasonic probe 20 such that the ultrasonic probe 20 is moved from the pre-movement position 621 to the post-movement position 622 while the imaging area of the ultrasonic probe 20 includes a major axis cross-section, so that the pre- and post-movement locations 623 and 624 of the PA signal generator 13 are tracked.

FIG. 14B is an ultrasonic wave image when the robot arm 90 is used to move the ultrasonic probe 20 like in FIG. 14A.

In the ultrasonic wave image, the blood vessel 82 of the subject 80 and the PA signal generator 13 (location 625) are depicted. This makes it easier to grasp the positional relationship between an stenosis lesion 83 and the PA signal generator 13 (location 625) when the stenosis lesion 83 is present in the blood vessel 82 of the subject 80.

In this regard, however, a way of moving the ultrasonic probe 20 is permitted if a reflected ultrasonic wave signal and a PA signal necessary for the formation of a display image can be acquired during the display image formation at step S405 in FIG. 4 or 10. This way is not limited to the way of movement in FIG. 13A or FIG. 14A.

As illustrated in FIG. 8, for instance, the location can be detected if both the speed of translation 711 described in FIG. 6 and the speed of rotation 721 described in FIG. 7 are not 0.

Then, the robot arm 90 operates and moves swingably the ultrasonic probe 20 such that the speed vₑ of the translation 711 and the angular velocity ω_{d} of the rotation of the ultrasonic probe 20 are set in formula (4). As a result, the translation 711 and the rotation 721 are not equal to 0 at the same time. This allows the location of the PA signal generator 13 to be detected constantly and continuously. Provided that v₀ and ω₀ are each a constant for adjusting the speed; and T represents a cycle of movement back and forth.

$$\begin{matrix}[ Formula 4 ] & \\ {v}_{e} = {v}_{0} cos 2 π \frac{t}{T} , {ω}_{d} = {ω}_{0} sin 2 π \frac{t}{T} & ( 4 )\end{matrix}$$

Hereinabove, the embodiments of the ultrasonic wave imaging apparatus and the catheterization support system in the invention have been described. However, the respective embodiments may be used in combination, if appropriate, as long as they are technically consistent, and such a combination should be included in the invention.

### REFERENCE SIGNS LIST

10 Ultrasonic wave-generating device
11 Guidewire (Body insertion instrument)
12 Optical fiber
13 PA signal generator (Photoacoustic ultrasonic wave generator) (Beacon)
20 Ultrasonic probe
30 Ultrasonic imaging module
31 Transmitter
32 Receiver
33 Input unit
34 Display unit
35 Memory
40 Controller
41 Signal-processing section
411 Reflected ultrasonic wave signal-processing unit
412 PA signal analyzing unit (Ultrasonic signal analyzing unit)
42 PA source location detection section
421 Relative position-detecting unit
422 Relative speed-measuring unit
423 Position filter
424 Absolute position-detecting unit (Beacon location-acquiring unit)
425 Probe speed-measuring unit (Probe position-acquiring unit)
43 Display image formation section
44 3D anatomical information
45 Operation planning section
51 Display image
52 Display image
80 Subject
90 Robot arm
91 Probe position/attitude sensor (Probe position-acquiring unit)
100 Medical support system

1. An ultrasonic wave imaging apparatus comprising:
an ultrasonic probe configured to irradiate a subject with an ultrasonic wave and receive a reflected wave of the ultrasonic wave and receive an ultrasonic wave from a beacon inserted into the subject;
a probe position-acquiring unit configured to acquire a 3D position and an orientation of the ultrasonic probe;
a beacon location-acquiring unit configured to determine a 3D location of the beacon from a relative location and a relative speed of the beacon relative to the ultrasonic probe as calculated from an ultrasonic wave image of the ultrasonic waves received at the ultrasonic probe and the 3D position and the orientation of the ultrasonic probe as acquired by the probe position-acquiring unit; and
a display image formation section configured to use the ultrasonic wave image of the ultrasonic waves received at the ultrasonic probe to form an image displayed on a display unit.

2. The ultrasonic wave imaging apparatus according to claim 1, wherein the ultrasonic probe is a linear array probe.

3. The ultrasonic wave imaging apparatus according to claim 2, wherein the beacon is inserted into a blood vessel of the subject and is moved along the blood vessel, and
the beacon location-acquiring unit is configured to determine the relative location of the beacon relative to the ultrasonic probe from at least one of changes in position of the beacon and the ultrasonic probe on an imaging plane of the ultrasonic probe or a change in rotational position of the imaging plane of the ultrasonic probe relative to the beacon.

4. The ultrasonic wave imaging apparatus according to claim 3, wherein the beacon location-acquiring unit is configured to reduce an error by filtering when the relative position of the beacon relative to the ultrasonic probe is determined.

5. The ultrasonic wave imaging apparatus according to claim 4, wherein in the beacon location-acquiring unit, the error is modeled for the filtering when the relative position of the beacon relative to the ultrasonic probe is determined.

6. The ultrasonic wave imaging apparatus according to claim 5, wherein in the beacon location-acquiring unit, the filtering is performed by inferring a statistically most likely state from an error model, the location relative to the ultrasonic probe and the change in position of the beacon, and the position and the change in position of the ultrasonic probe.

7. The ultrasonic wave imaging apparatus according to claim 4, further comprising
a robot arm for operating the ultrasonic probe,
wherein the probe position-acquiring unit is configured to set information about an operation position of the robot arm to the 3D position of the ultrasonic probe.

8. The ultrasonic wave imaging apparatus according to claim 7, wherein the robot arm tracks the beacon such that the beacon is recognized in a captured image of a transverse cross-section of the blood vessel imaged using the ultrasonic probe.

9. The ultrasonic wave imaging apparatus according to claim 7, wherein the robot arm tracks the beacon such that the beacon is recognized in a captured image of a major axis cross-section of the blood vessel imaged using the ultrasonic probe.

10. The ultrasonic wave imaging apparatus according to claim 7, wherein the robot arm tracks the beacon such that the beacon location-acquiring unit determines a relative location of the beacon relative to the ultrasonic probe from at least one of changes in position of the beacon and the ultrasonic probe on an imaging plane of the ultrasonic probe or a change in rotational position of an imaging plane of the ultrasonic probe relative to the beacon.

11. The ultrasonic wave imaging apparatus according to claim 2, wherein the display image formation section is configured to form a display image based on pre-acquired anatomical structure information about the subject and the 3D location of the beacon as determined in the beacon location-acquiring unit.

12. The ultrasonic wave imaging apparatus according to claim 11, wherein the display image formation section is configured to form the display image in view of in vivo deformation.

13. A medical support system comprising:
the ultrasonic wave imaging apparatus according to claim 1; and
a guidewire having a beacon at a tip thereof.

14. A method for displaying an image in an ultrasonic wave imaging apparatus including a linear array probe configured to irradiate a subject with an ultrasonic wave and receive a reflected wave of the ultrasonic wave and receive an ultrasonic wave from a beacon inserted into the subject, the method comprising:
acquiring a 3D position and an orientation of the linear array probe;
determining a relative location of the beacon relative to the linear array probe from an ultrasonic wave image received at the linear array probe;
determining a 3D location of the beacon from the relative location of the beacon and the 3D position and the orientation of the linear array probe;
operating, based on the 3D location of the beacon, the position of the linear array probe; and
displaying an ultrasonic wave image from the ultrasonic waves received at the linear array probe.
